(19) United States
(12) Patent Application Publication
(10) Pub. No.: US 2024/0161317 A1
(43) Pub. Date: May 16, 2024

(54) ENHANCING DENTAL VIDEO TO CT MODEL REGISTRATION AND AUGMENTED REALITY AIDED DENTAL TREATMENT

(71) Applicant: Mars Dental AI Ltd., Beit Yitzhak-Sha'ar Hefer (IL)

(72) Inventor: Ariel SHUSTERMAN, Kfar Hasidim (IL)

(73) Assignee: Mars Dental AI Ltd., Beit Yitzhak-Sha'ar Hefer (IL)

(21) Appl. No.: 18/280,723

(22) PCT Filed: Mar. 10, 2022
(86) PCT No.: PCT/IL2022/050274

§ 371 Date: Sep. 7, 2023

Related U.S. Application Data
(60) Provisional application No. 63/159,475, filed on Mar. 11, 2021.
(60) Provisional application No. 63/161,152, filed on Mar. 15, 2021.
(60) Provisional application No. 63/299,017, filed on Jan. 13, 2022.

(51) Int. Cl. G06T 7/33; G06T 7/00; G06T 19/00

(52) U.S. Cl. CPC G06T 7/344; G06T 7/0016; G06T 19/006; G06T 2207/10016; G06T 2207/10024; G06T 2207/10081; G06T 2207/20081; G06T 2207/30036; G06T 2207/30204

(57) ABSTRACT

Disclosed herein methods and systems for registering intraoral frames of a patient to a dental CT model of the patient, comprising receiving a dental 3D visual model of a patient created based on an intraoral scan of the patient captured after one or more teeth of the patient are marked with intraoral markers located inside the patient's mouth, registering the 3D visual model to a 3D CT model of the patient, receiving intraoral frames captured during a dental treatment to the patient, each frame depicts at least some of the intraoral markers, registering each frame to the 3D visual model based on the intraoral markers, registering each frame to the 3D CT model based on its registration to the 3D visual model registered to the 3D CT model, and creating a plurality of fused frames each merging a respective frame with a corresponding segment of the registered 3D CT model.

### RELATED APPLICATIONS

This application claims the benefit of priority under 35 USC § 119(e) of U.S. Provisional Patent Application Nos. 63/159,475 filed on Mar. 11, 2021, U.S. Provisional Patent Application No. 63/161,152 filed on Mar. 15, 2021 and U.S. Provisional Patent Application No. 63/299,017 filed on Jan. 13, 2022. The contents of the above applications are all incorporated by reference as if fully set forth herein in their entirety.

### BACKGROUND

The present invention, in some embodiments thereof, relates to registering intraoral frames captured during a dental treatment to a dental CT model, and, more specifically, but not exclusively, to registering intraoral frames captured during a dental treatment to a dental CT model based on intraoral markers marked on teeth of the patient inside his mouth and do not protrude out of the mouth.

Imaging technologies have dramatically evolved in recent times and have spread to numerous applications, uses and practices. Among other applications, the use of imaging to support dental procedures has also dramatically increased, in particular for more complex dental procedures such as, for example, dental surgery, dental implants and/or the like.

One such dental application is fusion of visual intraoral imagery data captured during the dental treatment (procedure) with Computed Tomography (CT) data which may be captured in advance using suitable equipment to form fused models presenting both visible dental features (e.g. teeth, gums, jaws, et.) as well as dental anatomy features (e.g. roots, root canals, etc.) which are typically invisible to the dental care givers during the dental treatment.

### SUMMARY

According to a first aspect of the present invention there is provided a method of registering intraoral frames of a patient to a dental Computed Tomography (CT) model of the patient, comprising using one or more processors configured for:
Receiving a dental 3D visual model of a patient created based on a visible light spectrum intraoral scan of the patient captured after one or more of a plurality of teeth of the patient are marked with a plurality of intraoral markers located with an intraoral space of the patient.
Registering the dental 3D visual model to a dental 3D CT model of the patient.
Receiving a plurality of frames captured during a dental treatment to the patient. Each of the plurality of frames depicts at least some of the plurality of intraoral markers.
Registering each frame to the dental 3D visual model based on the at least some intraoral markers.
Registering each frame to the dental 3D CT model based on the registration of the respective frame to the dental 3D visual model and the registration of the dental 3D visual model to the dental 3D CT model.
Creating a plurality of fused frames each merging a respective one of the plurality of frames with a corresponding segment of the registered dental 3D CT model.

According to a second aspect of the present invention there is provided a system for registering intraoral frames of a patient to a dental CT model of the patient, comprising one or more processors configured to execute a code. The code comprising:
code instructions to receive a dental 3D visual model of a patient created based on a visible light spectrum intraoral scan of the patient captured after one or more of a plurality of teeth of the patient are marked with a plurality of intraoral markers located with an intraoral space of the patient.
Code instructions to register the dental 3D visual model to a dental 3D CT model of the patient.
code instructions to receive a plurality of frames captured during a dental treatment to the patient. Each of the plurality of frames depicts at least some of the plurality of intraoral markers.
Code instructions to register each frame to the dental 3D visual model based on the at least some intraoral markers.
Code instructions to register each frame to the dental 3D CT model based on the registration of the respective frame to the dental 3D visual model and the registration of the dental 3D visual model to the dental 3D CT model.
Code instructions to create a plurality of fused frames each merging a respective one of the plurality of frames with a corresponding segment of the registered dental 3D CT model.

According to a third aspect of the present invention there is provided a method of registering intraoral frames of a patient to a dental CT model of the patient, comprising using one or more processors configured for:
Receiving a dental 3D CT model of a patient created based on a CT scan captured after one or more of a plurality of teeth of the patient are marked with a plurality of intraoral markers located with an intraoral space of the patient. The plurality of markers are applied using one or more materials having a density deviating by predefined value from the density of the plurality of teeth such that the plurality of intraoral markers are distinguishable from the plurality of teeth in the CT scan and are visualized accordingly in the dental 3D CT model.
Receiving a plurality of frames captured by one or more imaging sensors during a dental treatment to the patient, each of the plurality of frames depicts at least some of the plurality of intraoral markers which are visible to the one or more imaging sensors.
Registering each frame to the dental 3D CT model based on the at least some intraoral markers.
Creating a plurality of fused frames each merging a respective one of the plurality of frames with a corresponding segment of the registered dental 3D CT model.

According to a fourth aspect of the present invention there provided is a system for registering intraoral frames of a patient to a dental CT model of the patient, comprising one or more processors configured to execute a code. The code comprising:
Code instructions to receive a dental 3D CT model of a patient created based on a CT scan captured after one or more of a plurality of teeth of the patient are marked with a plurality of intraoral markers located with an intraoral space of the patient. The plurality of intraoral markers are applied using one or more materials having a density deviating by predefined value from the density of the plurality of teeth such that the plurality of intraoral markers are distinguishable from the plurality of teeth in the CT scan and are visualized accordingly in the dental 3D CT model.
Code instructions to receive a plurality of frames captured by one or more imaging sensors during a dental treatment to the patient, each of the plurality of frames depicts at least some of the plurality of intraoral markers which are visible to the one or more imaging sensors.
Code instructions to register each frame to the dental 3D CT model based on the at least some intraoral markers.
Code instructions to create a plurality of fused frames each merging a respective one of the plurality of frames with a corresponding segment of the registered dental 3D CT model.

According to a fifth aspect of the present invention there is provided a method of registering intraoral frames of a patient to a dental CT model of the patient, comprising using one or more processors configured for:
Obtaining one or more Machine Learning (ML) models trained to register intraoral images of a patient to a dental 3D CT model of the patient. The one or more ML models are trained with a plurality of intraoral images of the patient captured prior to a dental treatment to the patient.
Receiving a plurality of intraoral frames of the patient captured by one or more imaging sensors during a dental treatment to the patient.
Applying the one or more trained ML models to register each of the plurality of intraoral frames to the dental 3D CT model.
Creating a plurality of fused frames each merging a respective one of the plurality of intraoral frames with a corresponding segment of the registered dental 3D CT model.

According to a sixth aspect of the present invention there is provided a system for registering intraoral frames of a patient to a dental CT model of the patient, comprising one or more processors configured to execute a code. The code comprising:
Code instructions to obtain one or more Machine Learning (ML) models trained to register intraoral images of a patient to a dental 3D CT model of the patient. The one or more ML models are trained with a plurality of intraoral images of the patient captured prior to a dental treatment to the patient.
Code instructions to receive a plurality of intraoral frames of the patient captured by one or more imaging sensors during a dental treatment to the patient.
Code instructions to apply the one or more trained ML models to register each of the plurality of intraoral frames to the dental 3D CT model.
Code instructions to create a plurality of fused frames each merging a respective one of the plurality of intraoral frames with a corresponding segment of the registered dental 3D CT model.

According to a seventh aspect of the present invention there is provided a method of enhancing accuracy of an augmented reality (AR) scene, comprising using one or more processors configured for:
Receiving a 2D video stream depicting a region of interest (ROI) of a AR scene projected in 3D to a user by an AR display device. The video stream is captured by one or more imaging sensors deployed such that a view angle of the one or more imaging sensors are aligned with the view angle of the user's eyes.
Calculating one or more projection attributes for the video stream based on one or more parameters of the one or more imaging sensors.
Augmenting one or more frames of the video stream by inserting one or more synthetic objects. The one or more synthetic objects are positioned with respect to one or more real-world objects depicted in the one or more frames according to the least one projection attribute.
Adjusting the projected AR scene by injecting the augmented video stream into the AR display device such that the augmented video stream masks a corresponding section of the 3D AR scene.

According to an eight aspect of the present invention there is provided a system for enhancing accuracy of an augmented reality (AR) scene, comprising one or more processors configured to execute a code. The code comprising:
Code instructions to receive a 2D video stream depicting a region of interest (ROI) of a AR scene projected in 3D to a user by an AR display device. The video stream is captured by one or more imaging sensors deployed such that a view angle of the one or more imaging sensors are aligned with the view angle of the user's eyes.
Code instructions to calculate one or more projection attributes for the video stream based on one or more parameters of the one or more imaging sensors.
Code instructions to augment one or more frames of the video stream by inserting one or more synthetic objects, the one or more synthetic objects are positioned with respect to one or more real-world objects depicted in the one or more frames according to the least one projection attributes.
Code instructions to adjust the projected AR scene by injecting the augmented video stream into the AR display device such that the augmented video stream masks a corresponding section of the 3D AR scene.

In a further implementation form of the first and/or second aspects, the dental 3D CT model of the patient is created in advance before the one or more teeth are marked with the plurality of intraoral markers.

In a further implementation form of the first and/or second aspects, the dental 3D visual model is created based on an intraoral scan captured prior to the dental treatment after the one or more teeth are marked with the plurality of intraoral markers.

In a further implementation form of the first and/or second aspects, the dental 3D visual model is created based on an intraoral scan captured during the dental treatment.

In a further implementation form of the first, second, third and/or fourth aspects, the intraoral markers are visible in visible light spectral region detectable by one or more imaging sensors used during the dental treatment to capture the plurality of frames.

In a further implementation form of the first, second, third and/or fourth aspects, the plurality of intraoral markers are created on the one or more teeth using one or more materials approved for intra oral use.

In a further implementation form of the first, second, third, fourth, fifth and/or sixth aspects, the plurality of frames are black and white frames and/or color frames.

In a further implementation form of the first, second, third, fourth, fifth and/or sixth aspects, the plurality of frames comprise depth data.

In an optional implementation form of the first, second, third, fourth, fifth and/or sixth aspects, one or more of the plurality of frames are registered to the dental 3D CT model by registering the one or more frames to another frame previously registered to the dental 3D CT model.

In a further implementation form of the first, second, third, fourth, fifth and/or sixth aspects, the plurality of fused frames are displayed via one or more Augmented Reality (AR) devices used by a dental caregiver treating the patient.

In a further implementation form of the seventh and/or eighth aspects, the AR session is a dental AR session in which the AR scene is an intraoral AR scene of a patient.

In a further implementation form of the seventh and/or eighth aspects, the one or more parameters comprises one or more intrinsic parameters of the one or more imaging sensors, the one or more intrinsic parameter are members of a group comprising: a focal length, a sensor format, and a principal point.

In a further implementation form of the seventh and/or eighth aspects, the one or more parameters comprises one or more extrinsic parameters of the one or more imaging sensors, the one or more extrinsic parameter are members of a group comprising: a position of the one or more imaging sensor with respect to the user's eyes, and a field of view.

In an optional implementation form of the seventh and/or eighth aspects, one or more algorithms are applied to smooth an edge of the augmented video stream injected into the 3D AR scene.

In an optional implementation form of the seventh and/or eighth aspects, a zoom of one or more frames of the video stream is adjusted before augmenting it and injected it into the 3D AR scene.

In an optional implementation form of the seventh and/or eighth aspects, one or more attributes of one or more pixels of one or more frames of the video stream are adjusted before injecting it into the AR scene, the one or more attributes are members of a group consisting of: brightness, color, and contrast.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of an exemplary process of registering intraoral frames of a patient to a dental CT model of the patient based on intraoral markers, according to some embodiments of the present invention;

FIG. 2 is a schematic illustration of an exemplary system for registering intraoral frames of a patient to a dental 3D CT model of the patient based on intraoral markers, according to some embodiments of the present invention;

FIG. 3 presents images of exemplary intraoral markers used for registering intraoral frames of a patient to a dental 3D CT model of the patient, according to some embodiments of the present invention;

FIG. 4 presents schematics illustration of exemplary dental 3D visual models internal of a patient comprising intraoral markers applied on teeth of the patient, according to some embodiments of the present invention;

FIG. 5 presents schematics illustration of merging intraoral frames captured for a patient with an exemplary dental 3D CT model of the patient, according to some embodiments of the present invention;

FIGS. 6A and 6B present a legacy sequence of registering intraoral frames to a dental 3D CT model using an external marker vs. a sequence of registering the intraoral frames to the dental 3D CT model based on intraoral markers, according to some embodiments of the present invention;

FIG. 7 is a flowchart of another exemplary process of registering intraoral frames of a patient to a dental CT model of the patient based on intraoral markers, according to some embodiments of the present invention;

FIG. 8 is a flowchart of an exemplary process of registering intraoral frames of a patient to a dental CT model of the patient using trained ML model(s), according to some embodiments of the present invention;

FIG. 9 is a flowchart of an exemplary process of enhancing accuracy of an Augmented Reality (AR) scene, according to some embodiments of the present invention;

FIG. 10 is a schematic illustration of an exemplary system for enhancing accuracy of a dental AR scene, according to some embodiments of the present invention; and

FIG. 11A, FIG. 11B and FIG. 11C are schematic illustrations of an exemplary dental AR scene augmented to include by injecting an augmented video stream in which synthetic object is positioned accurately with respect to real-world objects depicted in the video stream, according to some embodiments of the present invention.

### DETAILED DESCRIPTION

The present invention, in some embodiments thereof, relates to registering intraoral frames captured during a dental treatment to a dental CT model, and, more specifically, but not exclusively, to registering intraoral frames captured during a dental treatment to a dental CT model based on intraoral markers marked on teeth of the patient inside his mouth and do not protrude out of the mouth.

Employing imaging technologies for dental treatment, planning and/or procedures has become wide spread, in particular for more complex dental procedures such as, for example, dental surgery, dental implants and/or the like.

One of the most prominent applications is fusing visual intraoral imagery data captured inside the intraoral space (mouth) of patients in visible light spectrum and thus depicting dental features visible to the dental caregivers (e.g. teeth, gums, jaws, etc.) with other imagery data, for example, Computed Tomography (CT) data which may depict dental anatomy features (e.g. roots, root canals, etc.) which are invisible to the dental care givers.

According to some embodiments of the present invention, there are provided methods, systems and computer software programs for registering intraoral frames (images) captured in the mouth (intraoral space) of a patient during a dental treatment to a dental 3 Dimensional (3D) CT model of the patient in order to create fused frames merging the intraoral frames with corresponding segments of the dental 3D CT model which are accurately positioned with respect to each other based on the accurate registration.

The fused frames which may depict both visible dental features and dental anatomy features extracted from the dental 3D CT model may be displayed to one or more caregivers treating the patient, for example, a dentist, an assistant and/or the like to provide them with an extensive intraoral view of the patient enriched with the invisible dental anatomy features.

In particular, since the intraoral frames are registered accurately to the dental 3D CT model, the position of each dental anatomy feature extracted from the dental 3D CT model may be accurately calculated with respect to the visible dental features thus providing the dental care giver an accurate view of the intraoral space of the patient which may enable them to assess, treat and/or operate the patient with increased assurance, safety and/or results.

The accurate registration is accomplished by first conducting an intraoral scan for the patient in the visible light spectrum after marking one or more of the teeth of the patient with intraoral markers which are internal (inside) in the intraoral space of the patient and do not protrude out of the patient's mouth. Based on the intraoral scan a dental 3D visual model may be created in which the intraoral markers are reflected (visible).

Using one or more model registration methods as known in the art, the dental 3D visual model may be registered to a dental 3D CT model created based on an intraoral CT scan conducted in advance (prior to the dental treatment) for the patient in one or more radiography spectral regions, for example, X-Ray, and/or the like.

During the dental treatment to the patient, a plurality of intraoral frames may be captured in the patient's mouth. Each of the frames may be registered accurately to the dental 3D visual model based on the intraoral markers detected in the respective intraoral frame compared to corresponding intraoral markers in the dental 3D visual model. Each of the frames may be then registered to the dental 3D CT model based on the registration of the dental 3D visual model to the dental 3D CT model.

After each frame is accurately registered to the dental 3D CT model, a position of one or more of the dental anatomy features extracted from the dental 3D CT model may be calculated accurately with respect to the intraoral features extracted and/or detected in the respective intraoral frame.

The fused frames may be created by merging the intraoral frames with corresponding sections of the dental 3D CT model thus depicting both the dental anatomy features and the visible intraoral features which are accurately positioned with respect to each other based on their respective calculated positions.

Using the intraoral markers for registering the intraoral frames to the dental 3D CT model may present major benefits and advantages compared to existing methods and systems used for registering the intraoral frames to the dental 3D CT model.

First, some of the existing methods register the intraoral frames captured during the fontal treatment to the dental 3D CT model created in advance based on an external marker that is detectible both in the intraoral frames and in the dental 3D CT model created based on an intraoral CT scan conducted in advance for the patient. In such methods the support elements, the external marker is detachably connected to one or more support elements installed in the mouth of the patient, for example, on one or more of his teeth prior to conducting the intraoral CT scan. While the external marker may be installed during the intraoral CT scan and during the dental treatment, the support element(s) are left in the mouth of the patient from before the CT scan until the end of the dental treatment. This time period may be significantly long since, typically the CT scan is conducted in advance of the dental treatment, for example, hours, days and/or weeks before the dental treatment. The patient is therefore forced to have the support element(s) installed in his mouth for a significantly long time which may be highly uncomfortable, irritating and possibly painful. This problem may be even more stressful in case the dental treatment stretches across a series of dental treatments over a certain period of time which may be significant.

Moreover, since the external marker is detached and re-attached to the support element(s) for the Ct scan and for the dental treatment, the position of the external marker may be different, even very slightly during the CT scan and during the dental treatment. As such registering the intraoral frames captured during the dental treatment to the dental 3D CT model created based on the intraoral CT scan may be inaccurate due to the different positions of the external marker.

Using the intraoral markers on the other hand completely removes these two limitations. First, the internal markers are applied only for the dental treatment since they are not required for the intraoral CT scan conducted for creating the dental 3D CT model. The time required for the patient to have the intraoral markers is therefore limited to the time of the dental treatment. However, even if the dental treatment is extended to several separate treatments over a certain period of the time, the patient may not feel any discomfort, irritation or pain since the intraoral markers may be marked such that they are not felt by the patient.

In addition, since the intraoral markers are not removed from the patient's teeth, altered and/or shifted in any way between the intraoral scan used to create the dental 3D visual model and the capturing of the intraoral frames, the registration of the intraoral frames to the dental 3D visual model may be highly accurate and since the dental 3D visual model is registered accurately to the dental 3D CT model using proved legacy methods, the registration of the intraoral frames to the dental 3D CT model may be highly accurate.

According to some embodiments of the present invention, the intraoral markers marked on one or more of the teeth of the patient may be visible in both the visible light spectrum and in the radiography spectral region(s) used for the intraoral CT scan. The intraoral markers may be therefore visible in both the dental 3D CT model created based on the intraoral CT scan and in the plurality of intraoral frames captured during the dental treatment to the patient.

In particular, the intraoral markers which may be marked using one or more materials having a density different from the density of the teeth and/or other dental anatomy features may be applied on the teeth of the patient prior to the intraoral CT scan conducted for creating the dental 3D CT model such that the intraoral markers may be visible in the intraoral CT scan and reflected (visible) accordingly in the dental 3D CT model created based on the intraoral CT scan.

Since the intraoral markers may be expressed in the dental 3D CT model and detected in the intraoral frames, the intraoral frames may be registered directly to the dental 3D CT model without requiring a dental 3D visual model thus eliminating the need for an intraoral scan conducted in the visible light spectrum for the patient 204.

Registering the intraoral frames to the dental 3D CT model based on the intraoral markers visible in both the visible light spectrum intraoral frames and in the radiography spectral region(s) based dental 3D CT model may present major benefits and advantages.

In particular, such registration may eliminate the need for the dental 3D visual model and thus eliminate the need for the intraoral scan. This may be highly advantageous since in some cases it may be impossible to perform the intraoral scan at the time of the dental treatment since the intraoral scan may require equipment and/or expertise not available to the dental caregiver conducting the dental treatment.

Moreover, eliminating the need for the intraoral scan and the dental 3D visual model may significantly reduce computing resource (e.g. processing resources, storage resources, etc.), time and/or effort otherwise required to produce them.

According to some embodiments of the present invention, the intraoral frames captured during the dental treatment may be registered to the dental 3D CT model created in advance using one or more ML models, for example, a neural network, a Deep Neural Network (DNN), a Support Vector Machine (SVM), and/or the like trained to detect dental features, for example, teeth, jaws, and/or the like visible in both the intraoral frames captured in the visible light spectrum and in the dental 3D CT model created based on an intraoral CT scan conducted in the radiography spectral region(s).

Using the ML model(s) for registering the intraoral frames to the dental 3D CT model may present major benefits and advantages. First, using the ML model(s) may remove the need for any type of marker for registering the intraoral frames captured during the dental treatment to the dental 3D CT model. Moreover, training the ML model(s) to register training intraoral frames captured in the intraoral space of each specific patient to the dental 3D CT model of the specific patient may yield ML model(s) highly customized and adapted for each specific patient. Such customized ML model(s) may be significantly more reliable and accurate compared to more general ML model(s) configured to support a plurality of patients as may be done by other methods which may be this significantly less reliable, accurate and/or robust.

According to some embodiments of the present invention, there are provided methods and systems for enhancing accuracy of Augmented Reality (AR) scenes projected to one or more users by one or more AR display devices, for example, an HMD, AR goggles, and/or the like during one or more AR sessions, for example, one or more dental AR sessions in which one or more dental caregivers may provide a dental treatment (procedure) to one or more patients.

Moreover, during the AR session one or more imaging sensors aligned with a line of sight of the user may capture a 2D video stream of a desired Region of Interest (ROI) in the AR scene.

One or more frames of the captured video stream may be augmented to include one or more synthetic objects placed in the video stream with respect to one or more real-world objects detected in the respective frame(s). Specifically, the synthetic objects may be positioned in the augmented frame(s) according to the one or more projection attributes of the frame(s) computed based on one or more operational parameters of the imaging sensor(s) in order to accurately position the synthetic object(s) in the augmented frame(s).

The augmented frames of the video stream may be then injected to the AR display device projecting the AR scene such that the augmented video stream masks (conceals, covers) a corresponding section of the AR scene displayed to the user.

Optionally, the edges of the augmented frame(s) in the AR scene may be smoothed in order to produce a finer and smoother AR scene in which the edges (borders) of the augmented frame(s) are significantly unnoticed. Optionally, a zoom of one or more of the frames of the video stream may be adjusted before injected into the AR scene. Optionally, one or more attributes of one or more pixels of one or more frames of the video stream, for example, brightness, color, contrast, and/or the like is adjusted before injected into the AR scene.

Enhancing the AR scene projected to users by AR display devices during AR sessions by augmenting the 2D video stream may present major benefits and advantages compared to existing AR methods and systems.

First, since the synthetic object(s) are inserted into the 2D video stream, there may be significantly less degrees of freedom for positioning the synthetic object(s) compared to the number of degrees of freedom existing in inserting synthetic object(s) to a 3D AR scene as may be done by the existing methods. The positioning of the synthetic object(s) in the frames of the video stream with respect to real-world object(s) depicted in the frames may be therefore significantly more accurate compared to positioning such synthetic object(s) in the 3D AR scene as may be done by existing methods and system.

Moreover, since both the synthetic object(s) and their reference real-world object(s) are displayed in the AR scene as part of the augmented video stream, the positioning of the synthetic object(s) with respect to the reference real-world is maintained constant as opposed to injecting 3D synthetic object(s) into the 3D AR scene as may be done by the existing methods where the position of the synthetic object(s) with respect to the real-world object(s) may change, shift and/or drift. Therefore, even if the augmented video stream is shifted with respect to the AR scene, the synthetic object(s) and their reference real-world object(s) which are part of the augmented video stream may not shift with respect to each other thus accurately positioned with respect to each other.

Furthermore, the real-world object(s) depicted in the video stream in association with the synthetic object(s) may be manipulated, for example, enlarged, reduced, brightened, darkened, sharpened, and/or the like. Such feature is obviously impossible in the existing methods where the real-world objects of the AR scene are directly perceived by the user.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

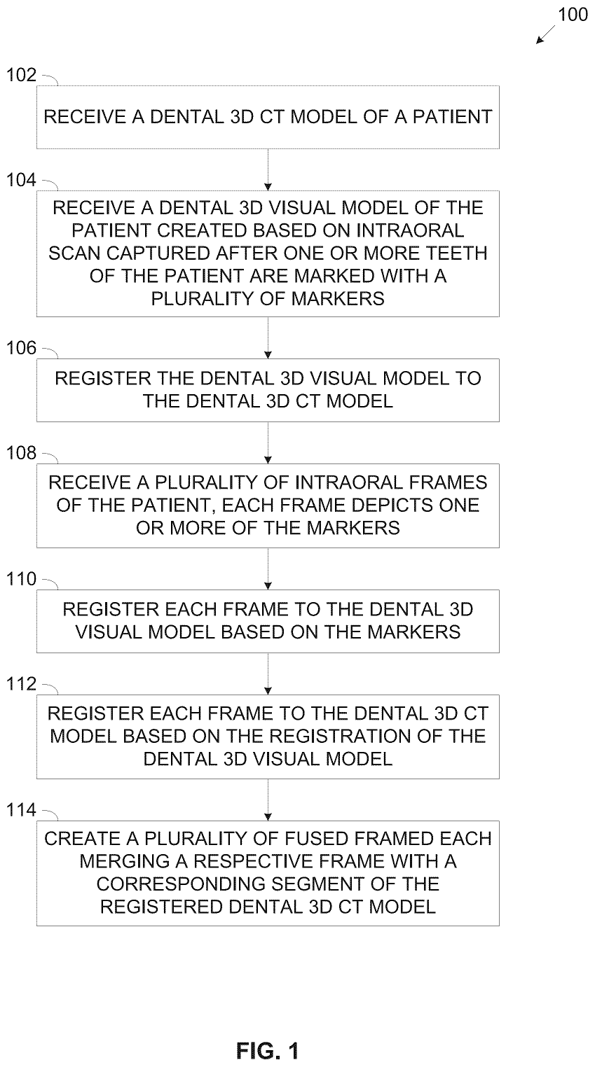

Referring now to the figures, FIG. 1 is a flowchart of an exemplary process of registering intraoral frames of a patient to a dental CT model of the patient based on intraoral markers, according to some embodiments of the present invention.

An exemplary process 100 may be executed to accurately register intraoral frames (images) captured in the mouth (intraoral space) of a patient during a dental treatment to a dental 3D CT model of the patient in order to create fused frames merging the intraoral frames with corresponding segments of the dental 3D CT model which are accurately positioned with respect to each other based on the accurate registration.

The fused frames may therefore combine dental anatomy features, for example, teeth roots, root canals, etc. extracted from the dental 3D CT model with the visible intraoral features depicted in the intraoral frames, for example, teeth, gums, jaw, and/or the like. The term visible as used herein throughout the description refers to the visible light spectrum (spectral region) visible to humans, for example, in the range of 350-750 nanometers.

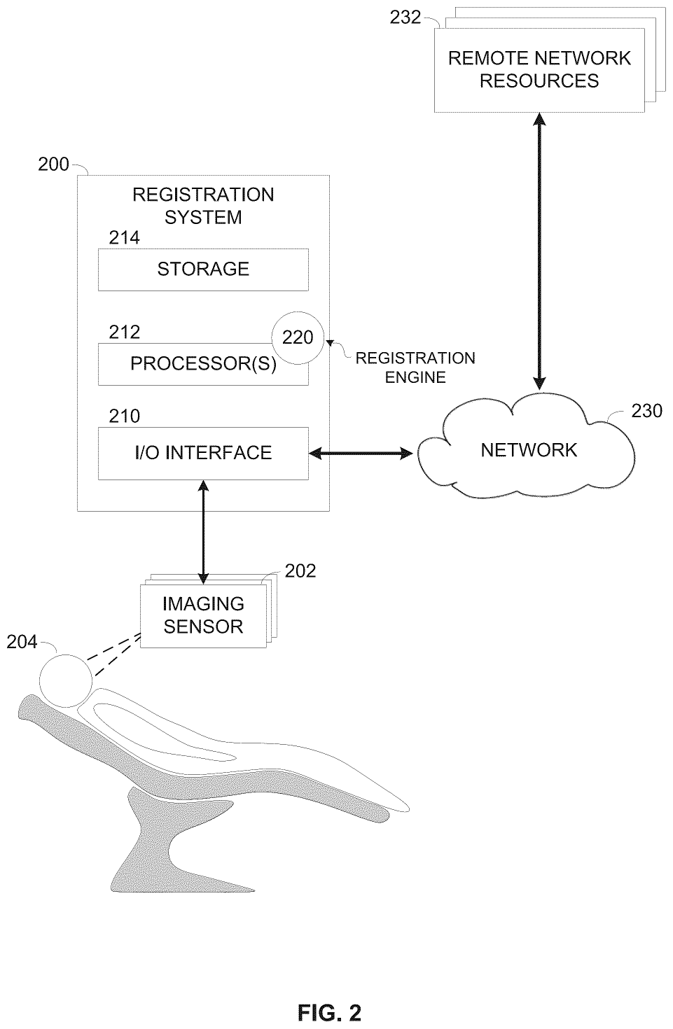

Reference is also made to FIG. 2, which is a schematic illustration of an exemplary system for registering intraoral frames of a patient to a dental 3D CT model of the patient based on intraoral markers, according to some embodiments of the present invention.

An exemplary registration system 200, for example, a computer, a server, a processing node, a cluster of processing nodes, and/or the like may be configured to execute the process 100 for registering intraoral frames captured by one or more imaging sensors 202, for example, a camera, a video camera, a stereoscopic camera, a depth camera, and/or the like during a dental treatment (dental procedure) to a patient 204, for example, a dental surgery, a dental implant, and/or the like.

The registration system 200 may comprise an Input/Output (I/O) interface 210 for connecting to the one or more imaging sensors 202, a processor(s) 212 for executing the process 100 and a storage 214 for storing data and/or code (program store).

The I/O interface 210 may include one or more wired and/or wireless I/O interfaces, for example, a Universal Serial Bus (USB) port, a serial port, a Bluetooth (BT) interface, a Radio Frequency (RF) interface, an infrared (IR) interface, a Near Field (NF) interface and/or the like. The I/O interface 210 may further include one or more wired and/or wireless network interfaces, for example, a Local Area Network (LAN) interface, a Wireless LAN (WLAN, e.g. Wi-Fi, etc.) interface, and/or the like.

Via the I/O interface 210, the registration system 200 may therefore connect and communicate with the imaging sensor(s) 202 to collect intraoral frames captured for the patient 204 during and optionally before the dental treatment. For example, assuming one or more of the imaging sensor(s) 202 supports Wi-Fi communications, the registration system 200 may communicate with this imaging sensor(s) 202 via the WLAN interface available in the I/O interface 210. In another example, assuming one or more of the imaging sensor(s) 202 are connectable via USB, the registration system 200 may communicate with this imaging sensor(s) 202 via the USB interface available in the I/O interface 210.

The I/O interface 210 may optionally include one or more additional wired and/or wireless network interfaces, for example, a Wide Area Network (WAN) interface, a Municipal Area Network (MAN) interface, a cellular interface and/or the like for connecting to a network 230 comprising one or more wired and/or wireless networks, for example, a LAN, a WAN, a MAN, a cellular network, the internet and/or the like. Via the I/O interface 210, the registration system 200 may communicate over the network 230 with one or more remote network resources, for example, a remote server, a remote storage resource, a cloud service, a cloud platform and/or the like, to receive and/or transmit data.

The processor(s) 212, homogenous or heterogeneous, may include one or more processing nodes and/or cores arranged for parallel processing, as clusters and/or as one or more multi core processor(s). The storage 214 may include one or more non-transitory persistent storage devices, for example, a Read Only Memory (ROM), a Flash array, a Solid State Drive (SSD), a hard drive (HDD) and/or the like. The storage 214 may also include one or more volatile devices, for example, a Random Access Memory (RAM) component, a cache and/or the like. The storage 214 may further comprise one or more attachable and/or network storage devices, for example, a storage server, a Network Accessible Storage (NAS), a network drive, a database server and/or the like accessible through the I/O interface 210.

The processor(s) 212 may execute one or more software modules such as, for example, a process, a script, an application, an agent, a utility, a tool, an Operating System (OS) and/or the like each comprising a plurality of program instructions stored in a non-transitory medium (program store) such as the storage 214 and executed by one or more processors such as the processor(s) 212. The processor(s) 212 may further, integrate, utilize and/or facilitate one or more hardware elements (modules) integrated and/or utilized in the registration system 200, for example, a circuit, a component, an Integrated Circuit (IC), an Application Specific Integrated Circuit (ASIC), a Field Programmable Gate Array (FPGA), a Digital Signals Processor (DSP), a Graphic Processing Unit (GPU), an Artificial Intelligence (AI) accelerator and/or the like.

The processor(s) 212 may therefore execute one or more functional modules implemented using one or more software modules, one or more of the hardware modules and/or combination thereof, for example, a registration engine 220 configured to execute the process 100 for registering intraoral frames of the patient 204 to the dental 3D CT model of the patient and crating merged frames combing visible intraoral features and invisible dental anatomy features of the patient 204.

For brevity, the process 100 and the system 200 are described for a single patient 204. This, however, should not be construed as limiting since the process 100 executed by the registration engine 220 may be repeated and expanded for a plurality of patients 204 each associated with a receptive dental 3D CT model and a respective dental 3D visual model.

As shown at 102, the process 100 starts with the registration engine 220 receiving a dental 3D CT model of the patient 204.

The dental 3D CT model may be created based on a radiography intraoral scan conducted in one or more spectral regions, for example, X-Ray, and/or the like. The dental 3D CT model may be therefore typically created for the patient 204 in advance before the patient 204 attends to the dental treatment since the radiography intraoral scan may require equipment and/or expertise not typically available to the dental caregivers (dentists) conducting the dental treatment to the patient 204.

The registration engine 220 may receive the dental 3D CT model via the I/O interface 210 from one or more storage locations, repositories and/or the like. For example, assuming the dental 3D CT model is stored in a USB attachable storage media (e.g. memory stick), the attachable storage media may be attached to the USB port of the I/O interface to enable the registration engine 220 to retrieve the stored dental 3D CT model. In another example, the registration engine 220 may receive the dental 3D CT model from one or more of the remote network resources 232 via the network 230.

As shown at 104, the registration engine 220 may receive a dental 3D visual model of the patient 204. In particular, the dental 3D visual models created based on an intraoral scan using one or more intraoral scanners as known in the art after marking one or more of the teeth of the patient 204 with intraoral markers.

The intraoral markers marked on the teeth of the patient 204 are located internally inside the mouth (intraoral space) of the patient 204 and do not protrude out of the patient's mouth. Moreover, the intraoral markers may be configured to avoid discomfort, unease and/or irritation to the patient 204.

The intraoral markers may include a plurality of intraoral markers such that frames captured by the imaging sensor(s) 202 in the mouth of the patient 204 may depict multiple intraoral markers which may be used to calculate accurately the position of imaging sensor 202 with respect to the intraoral markers using one or more algorithms, for example, triangulation and/or the like.

Moreover, the intraoral markers may be marked on one or more teeth of the patient 204 may be distributed on the teeth such that from each applicable view point from which the imaging sensor(s) 202 may be operated to capture the intraoral frames, multiple intraoral markers of the plurality of intraoral markers may be visible to the imaging sensor(s) 202 and thus detectable in the captured intraoral frames.

The intraoral markers may be marked on the teeth of the patient 204 using one or more materials visible in one or more spectral ranges detectable by the imaging sensors 202, in particular in the visible light spectral region. Moreover, the intraoral markers may be created (painted) using one or more materials approved for intra oral use, for example, composite resin, glass ionomer cement, compomers, dental cermets, and/or the like.

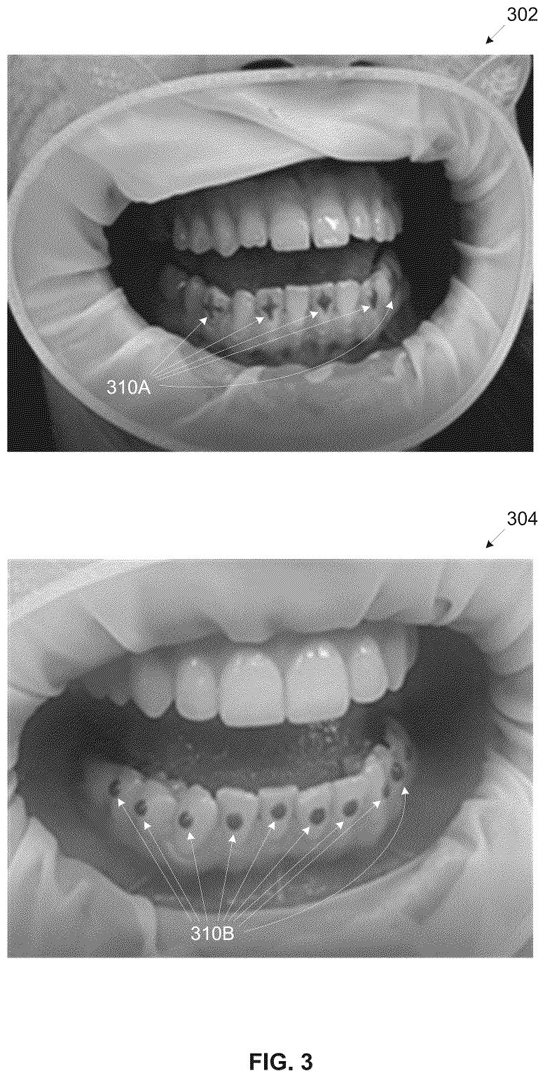

Reference is made to FIG. 3, which presents images of exemplary intraoral markers used for registering intraoral frames of a patient to a dental 3D CT model of the patient, according to some embodiments of the present invention.

As seen in image 302, a plurality of intraoral markers 310, for example, cross shapes 310A may be marked on a subset of the teeth of a patient such as the patient 204, for example, on alternating bottom teeth.

In another example, as seen in image 304, a plurality of intraoral markers 310, for example, dots 310B may be marked on a subset of the teeth of the patient 204, for example, on all bottom teeth.

As stated herein before, the dental 3D visual model is created based on the intraoral scan conducted as known in the art during which the intraoral environment of the patient 204 may be scanned and mapped. For example, light source laser, structured light and/or the like may be projected in the mouth of the patient and a plurality of images may be captured in a plurality of viewpoints, axes and/or positions to map the visible dental features in the mouth of the patient 204.

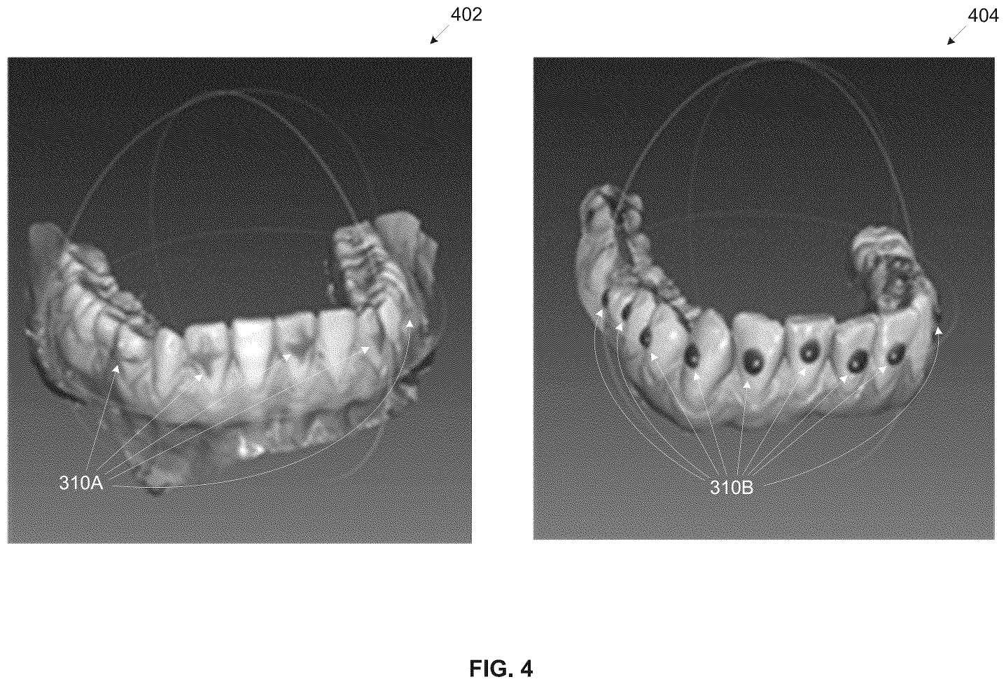

Reference is now made to FIG. 4, which presents schematics illustration of exemplary dental 3D visual models internal of a patient comprising intraoral markers applied on teeth of the patient, according to some embodiments of the present invention;

Exemplary dental 3D visual models 402 and 404 may be created based on intraoral scans captured to scan the mouth of one or more patients such as the patient 204. Since the dental 3D visual models 402 and 404 are created based on intraoral scan captured after intraoral markers are applied to the teeth of the patient(s) 204, the intraoral markers may be clearly visible in the dental 3D visual models 402 and 404. For example, the dental 3D visual models 402 is created based on an intraoral scan conducted to scan the mouth demonstrated in image 302 and the cross shapes 310A are therefore clearly visible in the dental 3D visual models 402. In another example, the dental 3D visual models 404 is created based on an intraoral scan conducted to scan the mouth demonstrated in image 304 and the dot shapes 310B are therefore clearly visible in the dental 3D visual models 404.

The intraoral scan used to create the dental 3D visual model may be captured prior to the dental treatment. For example, the intraoral markers may be marked on one or more teeth of the patient 204a certain time before the dental treatment, for example, one or more hours, one or more days and/or the like and the patient 204 may thus undergo the intraoral scan before the dental treatment. This may be suitable for cases where the intraoral scan may be done at a different location and/or by different personnel than that conducting the dental treatment.

However, intraoral scan used to create the dental 3D visual model may be captured during the dental treatment, specifically at the time of the dental treatment before staring the actual dental procedure (e.g. surgery, implant, etc.). in such case, the patient 204 may arrive to receive the dental treatment and the intraoral markers may be marked on one or more of his teeth. The intraoral scan may be then conducted and the dental 3D visual model may be created based on the intraoral scan such that the dental 3D visual model is available for the actual dental procedure.

Optionally, the intraoral scan may be captured by one or more of the imaging sensor(s) 202 which may comprise an intraoral scanner.

The dental 3D visual model may be created by the registration system 200, for example, the registration engine 220 and/or by one or more different systems. In case the dental 3D visual model is created locally by the registration engine 220 based on an intraoral scan conducted elsewhere, the registration engine 220 may receive the intraoral scan via one or more attachable storage media attached to the I/O interface 210 and/or from one or more of the remote network resources 232 via the network 230. In case, the dental 3D visual model is created by different system(s), the registration engine 220 may receive the dental 3D visual model via one or more attachable storage media attached to the I/O interface 210 and/or remote network resources 232 via the network 230.

Reference is made once again to FIG. 1.

As shown at 106, the registration engine 220 may register the dental 3D visual model to the dental 3D CT model, i.e. map (transform) the dental 3D visual model and the dental 3D CT model to a common coordinate system.

The registration engine 220 may apply one or more one or more registration methods, techniques and/or algorithms as known in the art for registering the two 3D models to each other, for example, intensity-based algorithms, feature-based algorithms, transformation models and/or the like applied using computer vision, ML and/or the like.

After registering the dental 3D visual model to the dental 3D CT model, the registration engine 220 may calculate a relative position of each intraoral marker visible in the dental 3D visual model with respect to each of one or more of the dental anatomy features visible in the dental 3D CT model.

As shown at 108, the registration engine 220 may receive a plurality of intraoral frames, optionally a video stream of a plurality of intraoral frames captured during the dental treatment by the imaging sensor(s) 202 to scan and depict the mouth of the patient 204, i.e., the intraoral dental environment of the patient 204.

The intraoral frames captured by the imaging sensor(s) 202 may be characterized by one or more attributes typically according to the capabilities, settings and/or parameters of the imaging sensor(s) 202. For example, the intraoral frames may comprise one or more black and white frames, one or more grey scale frames, one or more color frames, and/or the like. In another example, one or more of the intraoral frames, for example, frames captured by a depth camera and/or a stereoscopic camera may comprise depth data.

Since, as described herein before, the intraoral markers are marked on the teeth of the patient 204 such that one or more of the intraoral markers, preferable multiple intraoral markers are visible from any viewpoint of the imaging sensor(s) 202, each of the intraoral frames may depict one or more of the intraoral markers, typically multiple markers.

As shown at 110, the registration engine 220 may register each received intraoral frame to the dental 3D visual model based on at least some of the intraoral markers detected in the respective intraoral frame compared to corresponding intraoral markers defined in the dental 3D visual model.

Registering each intraoral frame may typically comprise mapping (transforming) the respective intraoral frame and the dental 3D visual model to a common coordinate system. The registration engine 220 may apply one or more methods, techniques and/or algorithms as known in the art for registering (aligning) the intraoral frames to the dental 3D visual model based on the intraoral markers, for example, intensity-based algorithms, feature-based algorithms, transformation models to name a few which may be conducted using computed vision, ML and/or the like.

As shown at 112, based on the registration of the dental 3D visual model to the dental 3D CT model and the registration of each intraoral frame the dental 3D visual model, the registration engine 220 may register accurately each of the intraoral frame to the dental 3D CT model.

Optionally, the registration engine 220 may register one or more of the intraoral frames to the dental 3D CT model indirectly by registering the respective intraoral frame to one or more other intraoral frames previously registered to the dental 3D CT model. For example, the registration engine 220 may apply one or more tracking algorithms to track one or more objects, for example, visible dental features in a plurality of subsequent intraoral frames and may thus register the later intraoral frames based on registration of earlier intraoral frame(s) to the dental 3D CT model.

Based on the accurate registration of each intraoral frame to the dental 3D CT model, the registration engine 220 may calculate accurately a position of each intraoral marker visible in each intraoral frame with respect to each of one or more of the dental anatomy features visible in the dental 3D CT model.

As shown at 114, the registration engine 220 may create a plurality of fused frames each merging a respective one of the intraoral frames with a corresponding segment of the dental 3D CT model.

In particular, since each intraoral frame is accurately registered to the dental 3D CT model and the position of each dental anatomy features visible in the dental 3D CT model with respect to the intraoral markers visible in the respective intraoral frame may be accurately calculated, the respective fused frame created based on the respective intraoral frame may show dental anatomy features extracted from the dental 3D CT model accurately positioned with respect to the intraoral markers in the respective fused frame and hence accurately positioned with respect to the visible dental features seen in the respective fused frame.

Moreover, in case the intraoral frames comprise depth data, the fused frames may be 3D frames as they merge the depth containing intraoral frames with the 3D dental anatomy features extracted from the dental 3D CT model.

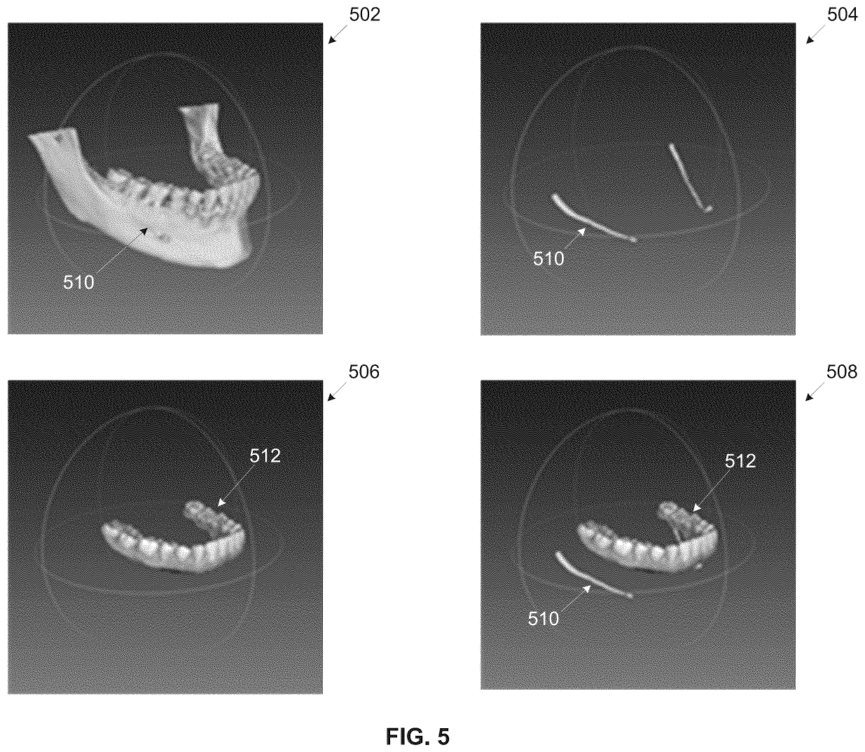

Reference is now made to FIG. 5, which presents schematics illustration of merging intraoral frames captured for a patient with an exemplary dental 3D CT model of the patient, according to some embodiments of the present invention.

As seen in 502, an exemplary dental 3D CT model may define a plurality of dental anatomy features, for example, teeth roots, root canals, and/or the like. As seen in 504, one or more dental anatomy features may be extracted from the dental 3D CT model, for example, a root canal 510. As seen in 506, an intraoral frame may depict one or more visible dental features, for example, a lower teeth set 512.

As seen in 508, a registration engine such as the registration engine 220 may create a fused frame merging the visible dental features detected in the intraoral frame 506, for example, the lower teeth set 512 with one or more of the dental anatomy features detected in the corresponding section of the dental 3D CT model, for example, the root canal 510. In particular, the registration engine 220 may create the fused frame 508 after accurately computing the position of the root canal 510 with respect to the intraoral markers detected in the intraoral frame 506 which is done as described herein before, based on the relative position of the intraoral markers detected in the dental 3D visual model to the dental anatomy features detected in the registered dental 3D CT model.

The plurality of fused frames may be displayed to the dental caregivers which may use the fused frames to explore, view and/or identify one or more of the dental features as well as the dental anatomy features of the patient 204 before and/or during the dental treatment (procedure).

Optionally, one or more of the plurality of fused frames are displayed to the dental caregivers via one or more Augmented Reality (AR) display devices used by a dental caregiver(s) treating the patient 204, for example, a Head Mounted Display (HMD), AR goggles, and/or the like. Moreover, in case the fused frames are 3D frames comprising depth data, the 3D fused frames displayed via the AR device(s) may significantly increase the increase the ability of the dental caregivers to identify the dental anatomy features with increased accuracy thus improving the dental treatment, for example, accurately placing a dental implant, accurately accessing a root canal, and/or the like.

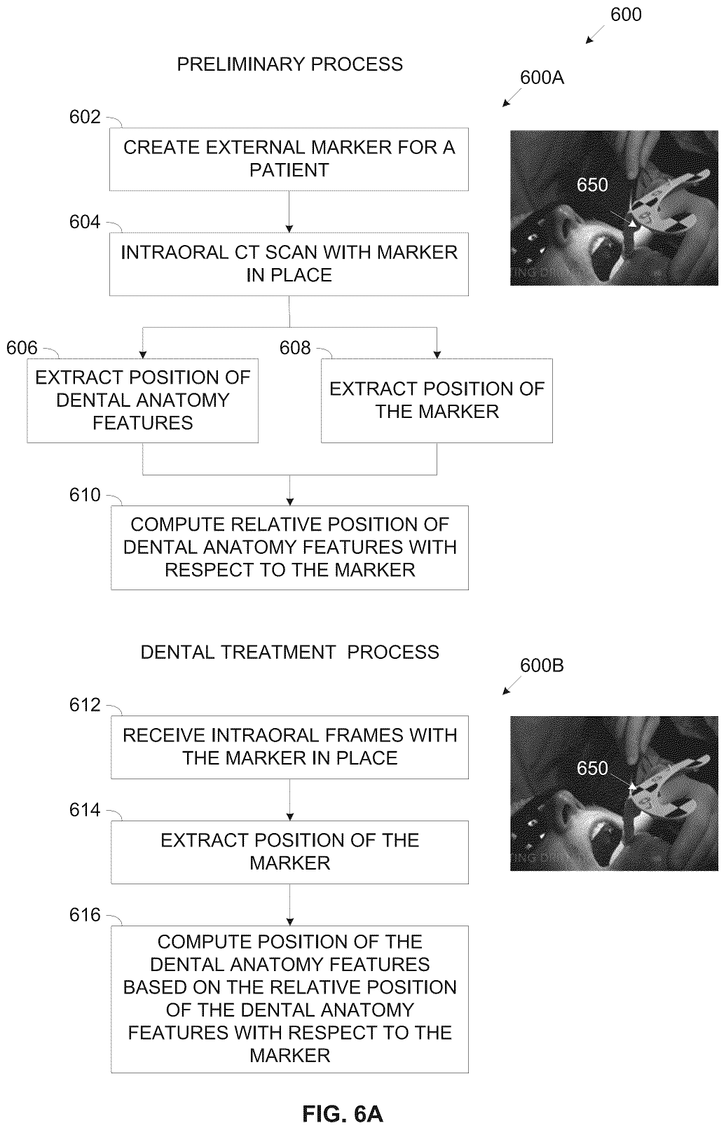

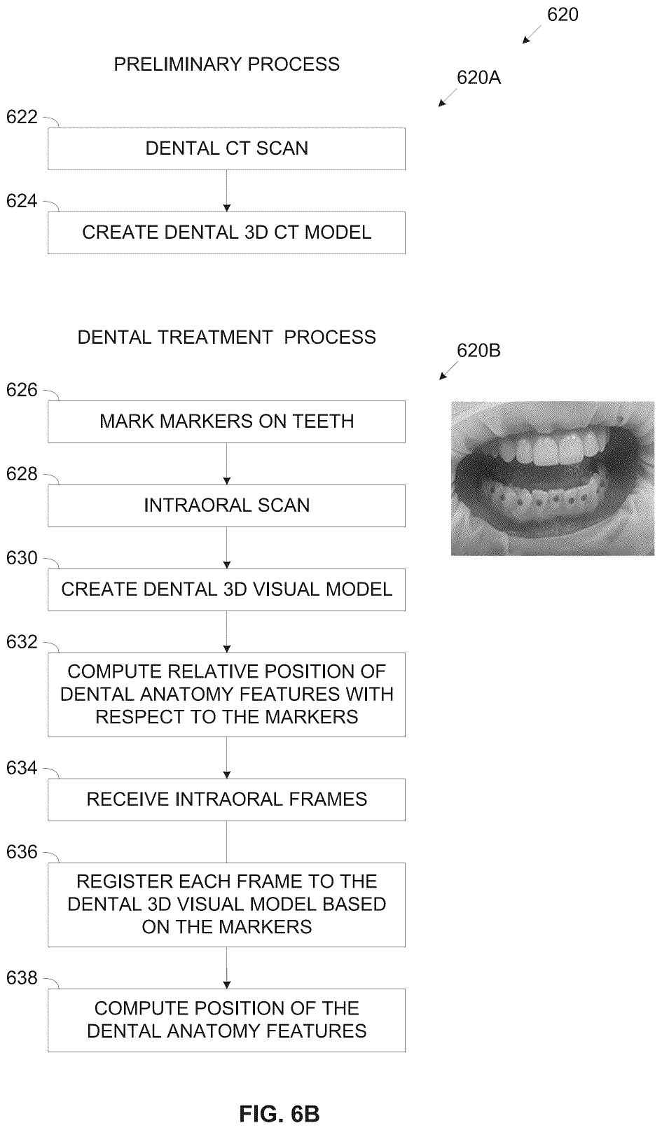

Reference is now made to FIGS. 6A and 6B, which present a legacy sequence of registering intraoral frames to a dental 3D CT model using an external marker vs. a sequence of registering the intraoral frames to the dental 3D CT model based on intraoral markers, according to some embodiments of the present invention.

As seen in FIG. 6A, an exemplary sequence 600 is a legacy sequence executed for registering intraoral frames captured in the intraoral space of a patient such as the patient 204 to a dental 3D CT model based on an external marker 650. The sequence 600 comprises two sub-sequences, a first preliminary process 600A comprising steps 602-610 conducted in advance prior to a second process 600B comprising steps 612-616 which is the actual dental treatment to the patient 204 (real-time).

As seen the external marker 650 is used in both the process 600A during which an intraoral CT scan is conducted to create a dental 3D CT model for the patient 204 and in the dental treatment process 600B. However, the preliminary process 600A may be conducted hours, days or even weeks before the dental treatment process 600B since the intraoral CT scan may be conducted using equipment, expertise and/or capabilities typically unavailable to the dental caregiver conducting the dental treatment 600B.

During the dental treatment 600B the dental caregiver may use imagery data (e.g. intraoral frames, intraoral vide, etc.) that must be aligned and synchronized to the dental 3D CT model, the imagery data may be registered to the dental 3D CT model based on the external marker 650.

Due to the significant time gap between the preliminary process 600A and the dental treatment 600B, the external marker 650 may be typically configured to detachably connect to a support element installed in the mouth of the patient 204, for example, connected to one or more teeth of the patient 204. The support element may be therefore left in the mouth of the patient 204 while the external marker 650 itself may be detached and removed from the patient 204. As such, the external marker may be located substantially similarly during the preliminary process 600A and the dental treatment 600B due to the fixed support element which is not removed between the preliminary process 600A and the dental treatment 600B. However, the external marker 650 itself may be removed from the patient 204 after completing the preliminary process 600A and reinstalled for the dental treatment process 600B to relief the patient 204 of the unease and complication involved in having the external marker 650 installed for a long period of time.

As seen at 602, the preliminary process 600A starts with creating the external marker 650 and installing it for the patient 204. As described herein before, this step may include installing the support element in the mouth of the patient 204 and attaching the external marker 650 to the fixed support element.

As seen at 604, the intraoral CT scan may be conducted for the patient 204 using one or more CT scanners configured to scan the intraoral space (mouth) of the patient 204 in one or more radiography spectral regions, for example, X-Ray and/or the like. Specifically, the intraoral CT scan is conducted while the external marker 650 is in place such that the intraoral CT scan may capture the external marker 650 in addition to the dental anatomy features of the patient 204.

As seen in 606, a position of one or more dental anatomy features may be extracted from the intraoral CT scan and as seen in 608, the position of the external marker 650 may be also extracted from the intraoral CT scan.

As seen in 610, a relative position may be computed for each of the dental anatomy features with respect to the external marker 650. Moreover, relative positions may be described, logged and/or presented in a dental 3D CT model created for the patient 204 based on the intraoral CT scan.

As seen at 612, during the dental treatment process 600B, a plurality of intraoral frames may be captured in the intraoral space of the patient 204 to support the dental caregiver in treating and/or planning the dental treatment to the patient 204. In particular, the intraoral frames which are captured in the visible light spectrum are captured after installing the external marker 650, i.e., connecting the external marker 650 to the support element installed in the mouth of the patient 204.

As seen at 614, the position of the marker is extracted from the intraoral frames and, as seen at 616, the position of the one or more of the dental anatomy features extracted from the dental 3D CT model may be calculated with respect to the external marker 650. This means, that the dental anatomy features may be positioned with respect to visible dental features of the patient 204 detected in the intraoral frames.

As seen in FIG. 6B, an exemplary process 620 for registering intraoral frames captured during a dental treatment of the patient 204 to a dental 3D CT model of the patient 204 according to some embodiments of the present invention, may also comprises two sub-sequences, a first preliminary process 620A comprising steps 622-624 conducted in advance prior to a second process 620B comprising steps 626-638 which is the actual dental treatment to the patient 204 (real-time).

As seen at 622, the preliminary process 600A starts with conducting an intraoral CT scan for the patient 204 and as seen at 624, a dental 3D CT model may be created for the patient 204 based on the intraoral CT scan.

As seen at 622, during the dental treatment process 600B, a plurality of intraoral markers may be marked on one or more of the teeth of the patient 204. As stated herein before, the intraoral markers are internal (inside) in the mouth of the patient 204 and do not protrude out of the patient's mouth.

As seen at 628, an intraoral scan may be conducted for the pertain 204 using one or more intraoral scanners as known in the art configured to operate in the visible light spectrum, and as seen in 630 a dental 3D visual model may be created for the patient 204 based on the intraoral scan.

As seen at 632, the position of the one or more dental anatomy features extracted from the intraoral CT scan and/or from the dental 3D CT model may be positioned with respect to the markers. In particular, the dental 3D visual model may be first registered to the dental 3D CT model and after properly registered to each other, the position of the dental anatomy features extracted from the dental 3D CT model may be accurately calculated with respect to the markers extracted from the dental 3D visual model.

As seen at 634, a plurality of intraoral frames may be captured in the intraoral space of the patient 204 to support the dental caregiver, and a seen at 636, each of the intraoral frames may be registered to the dental 3D visual model based on the intraoral markers detected in the respective intraoral frame compared to the intraoral markers described in the dental 3D visual model as described herein before in step 110 of the process 100.

As seen at 638, after registering each intraoral frame to the dental 3D visual model the position of one or more of the dental anatomy features extracted from the dental 3D CT model previously calculated with respect to the markers portrayed in the dental 3D visual model may be accurately calculated with respect to the markers detected in the respective intraoral frame.

Moreover, the dental anatomy feature(s) may be added to one or more of the intraoral frames in its calculated position thus creating a fused frame enriching the visual perspective of the intraoral environment of the patient 204 to the dental caregiver.

According to some embodiments of the present invention, the intraoral markers marked on one or more of the teeth of the patient 204 may be visible in both the visible light spectrum and in the spectral regions used for the intraoral CT scan. In such embodiments, the intraoral markers may be visible in the dental 3D CT model created based on the intraoral CT scan and in the plurality of intraoral frames captured during the dental treatment to the patient 204.

In such embodiments the intraoral markers marked on one or more of the teeth of the patient may be applied prior to the intraoral CT scan conducted for creating the dental 3D CT model. In particular, the intraoral markers may be marked using one or more materials having a density different from the density of the teeth and/or other dental anatomy features such that the intraoral markers may be visible in the CT scan such that the intraoral markers may be expressed, described and/or present in the dental 3D CT model.

Since the intraoral markers may be expressed in the dental 3D CT model and detected in the intraoral frames, the intraoral frames may be registered directly to the dental 3D CT model without requiring a dental 3D visual model thus eliminating the need for an intraoral scan conducted in the visible light spectrum for the patient 204.

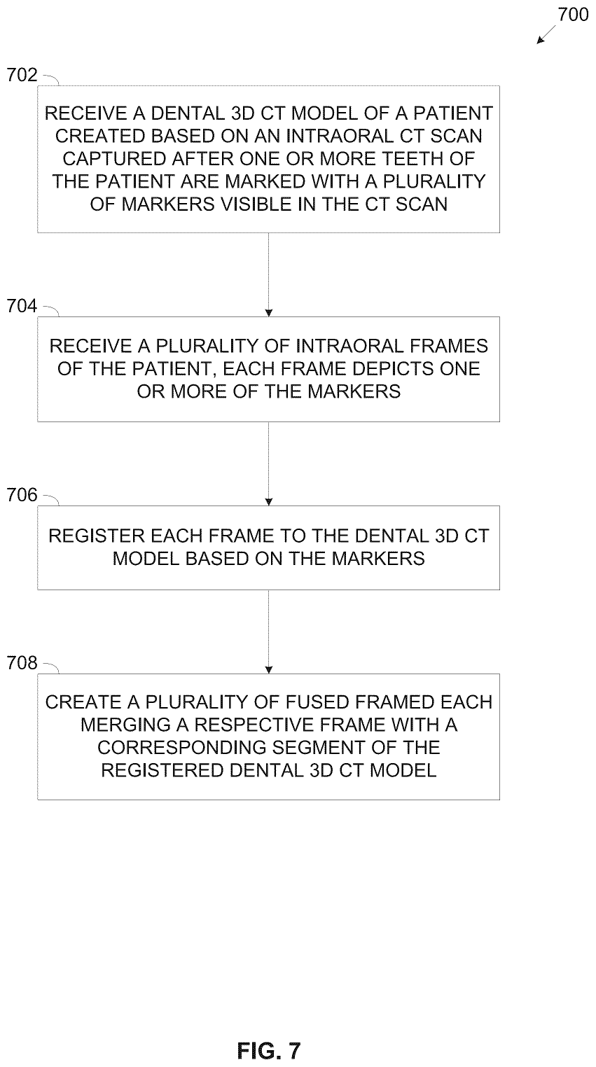

Reference is now made to FIG. 7, which is a flowchart of another exemplary process of registering intraoral frames of a patient to a dental CT model of the patient based on intraoral markers, according to some embodiments of the present invention.

An exemplary process 700 may be executed by a registration engine such as the registration engine 220 executed by a registration system such as the registration system 200 for registering a plurality of intraoral frames captured by one or more imaging sensors such as the imaging sensor 202 during a dental treatment of a patient such as the patient 204 to a dental 3D CT model created in advance for the patient 204.

For brevity, the process 700 and the system 200 are described for a single patient 204. This, however, should not be construed as limiting since the process 700 executed by the registration engine 220 may be repeated and expanded for a plurality of patients 204 each associated with a receptive dental 3D CT model.

As shown at 702, the process 700 starts with the registration engine 220 receiving a dental 3D CT model of the patient 204. Specifically, the dental 3D CT model may be created based on a radiography intraoral scan conducted for the patient 204 after a plurality of intraoral markers are marked on one or more teeth of the patient 204.

The intraoral markers may be marked using one or more materials characterized by having a density that deviates by more than predefined value (e.g., 20%, 25%, 30%, etc.) from the density of the plurality of teeth such that the intraoral markers are distinguishable from the plurality of teeth in the intraoral CT scan conducted in one or more radiography spectral regions, for example, X-Ray, and/or the like. While visible in the radiography spectral regions, the intraoral markers are also visible in the visible light spectrum.

Moreover, the intraoral may be marked and distributed on the teeth of the patient 204 as described in the step 104 of the process 100 such that multiple intraoral markers may be seen from any applicable view point from which the imaging sensor(s) 202 may be operated to capture the intraoral frames.

As it is created based on the intraoral CT scan in which the intraoral markers are visible, the dental 3D CT model may express, describe and/or present the intraoral markers.

As shown at 704, the registration engine 220 may receive a plurality of intraoral frames captured by the imaging sensor(s) 202 during the dental treatment as described in step 108 of the process 100. As described herein before, due to the distribution of the intraoral markers on the teeth of the patient 204, each of the received intraoral frames may depict one or more and preferably multiple intraoral markers.

As shown at 706, the registration engine 220 may register each received intraoral frame to the dental 3D CT model based on at least some of the intraoral markers detected in the respective intraoral frame compared to corresponding intraoral markers defined in the dental 3D CT model.

The registration engine 220 may apply one or more methods, techniques and/or algorithms as known in the art for registering (aligning) the intraoral frames to the dental 3D CT model based on the intraoral markers as described herein before in step 110 of the process 100 and transforming the intraoral frames and the dental 3D CT model to a common coordinate system.

Optionally, the registration engine 220 may register one or more of the intraoral frames to the dental 3D CT model indirectly by registering the respective intraoral frame to one or more other intraoral frames previously registered to the dental 3D CT model as described in the step 112 of the process 100.

Based on the accurate registration of each intraoral frame to the dental 3D CT model, the registration engine 220 may calculate accurately a position of each intraoral marker visible in each intraoral frame with respect to each of one or more of the dental anatomy features visible in the dental 3D CT model.

As shown at 708, the registration engine 220 may create a plurality of fused frames each merging a respective one of the intraoral frames with a corresponding segment of the dental 3D CT model as described in step 114 of the process 100 which may be displayed to one or more dental caregivers treating the patient 204.

According to some embodiments of the present invention, the intraoral frames captured during the dental treatment may be registered to the dental 3D CT model created in advance using one or more ML models, for example, a neural network, a Deep Neural Network (DNN), a Support Vector Machine (SVM), and/or the like trained to detect dental features, for example, teeth, jaws, and/or the like visible in both the intraoral frames captured in the visible light spectrum and in the dental 3D CT model created based on an intraoral CT scan conducted in the radiography spectral region(s).

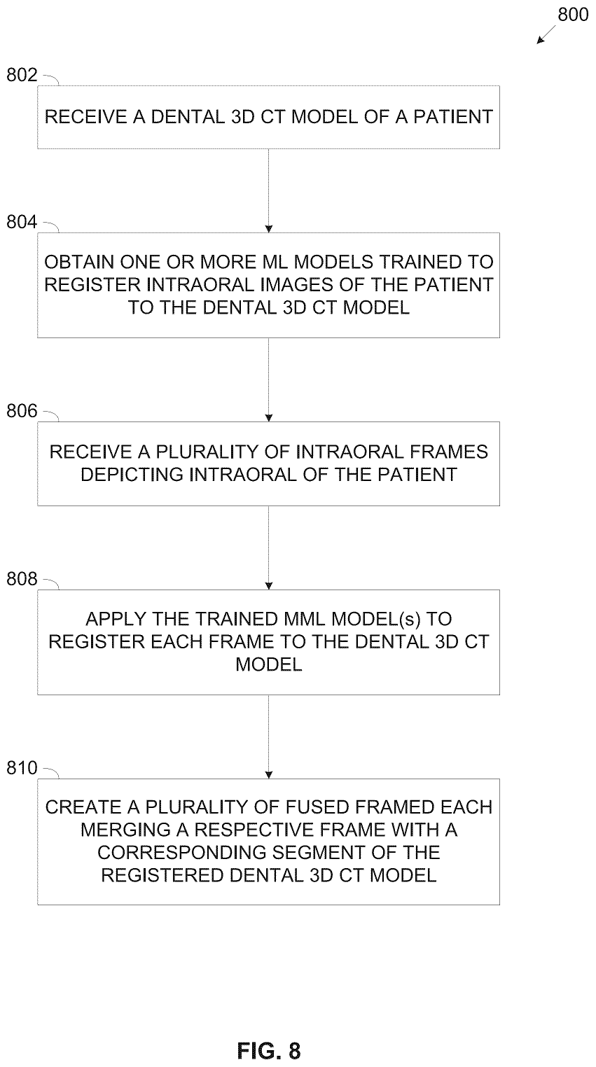

Reference is now made to FIG. 8, which is a flowchart of an exemplary process of registering intraoral frames of a patient to a dental CT model of the patient using trained ML model(s), according to some embodiments of the present invention.

An exemplary process 800 may be executed by a registration engine such as the registration engine 220 executed by a registration system such as the registration system 200 for registering a plurality of intraoral frames captured by one or more imaging sensors such as the imaging sensor 202 during a dental treatment of a patient such as the patient 204 to a dental 3D CT model created in advance for the patient 204.

As before, for brevity, the process 800 and the system 200 are described for a single patient 204. This, however, should not be construed as limiting since the process 800 executed by the registration engine 220 may be repeated and expanded for a plurality of patients 204 each associated with a receptive dental 3D CT model.

As shown at 802, the registration engine 220 may receive a dental 3D CT model of a patient 204 as described ion step 102 of the process 100.

As shown at 804, the registration engine 220 may obtain one or more ML models, for example, a neural network, a DNN, an SVM, and/or the like trained to register intraoral frames (images) captured in the intraoral space of the patient 204 to the dental 3D CT model.

The ML model(s) may be trained in one or more training sessions using a one or more training datasets comprising a plurality of training intraoral frames of the patient 204 registered to the dental 3D CT model of the patient 204. The training intraoral frames may be captured in the intraoral space of the patient 204 using one or more imaging sensors such as the imaging sensor 202.

The ML model(s) applied to the dental 3D CT model and to the training intraoral frames may detect a plurality of dental features, for example, teeth, jaws, and/or the like which are visible at least partially in both the training intraoral frames captured in the visible light spectrum and in the dental 3D CT model visualizing the intraoral space scanned in the radiography spectral region(s).

The ML model(s) may therefore evolve, adapt, adjust and/or learn, for example, adjust their internal paths, edges' weights and/or layers' structure to accurately register the training intraoral frames to the dental 3D CT model.

As shown at 806, the registration engine 220 may receive a plurality of intraoral frames captured by the imaging sensor(s) 202 during the dental treatment as described in step 108 of the process 100.

As shown at 808, the registration engine 220 may apply the trained ML model(s) to each of the received intraoral frames in order to register each intraoral frame top the dental 3D CT model of the patient 204.

As shown at 810, the registration engine 220 may create a plurality of fused frames each merging a respective one of the intraoral frames with a corresponding segment of the dental 3D CT model as described in step 114 of the process 100 which may be displayed to one or more dental caregivers treating the patient 204.

According to some embodiments of the present invention, there are provided methods and systems for enhancing accuracy of AR scenes perceived by one or more users wearing one or more AR display devices, for example, an HMD, AR goggles, and/or the like during one or more AR sessions.

In some exemplary embodiments, the AR sessions may comprise one or more AR dental sessions in which one or more dental caregivers may provide a dental treatment (procedure), for example, a dental surgery, a dental implant and/or the like to one or more patients such as the patient 204.

During the AR session one or more imaging sensors such as the imaging sensor 202, in particular, imaging sensor(s) which are aligned with a line of sight of the user may capture a 2D video stream of a desired Region of Interest (ROI) in the 3D AR scene.

One or more frames of the captured video stream may be augmented to include one or more synthetic objects placed in the video stream with respect to one or more real-world objects detected in the respective frame(s). In particular, the synthetic objects may be positioned in the augmented frame(s) according to the one or more projection attributes of the frame(s) computed based on one or more operational parameters of the imaging sensor(s) which captured the video stream.

The augmented video stream may be then injected to the AR display device projecting the 3D AR scene to the user such that the augmented video stream masks a corresponding section of the AR scene displayed to the user.

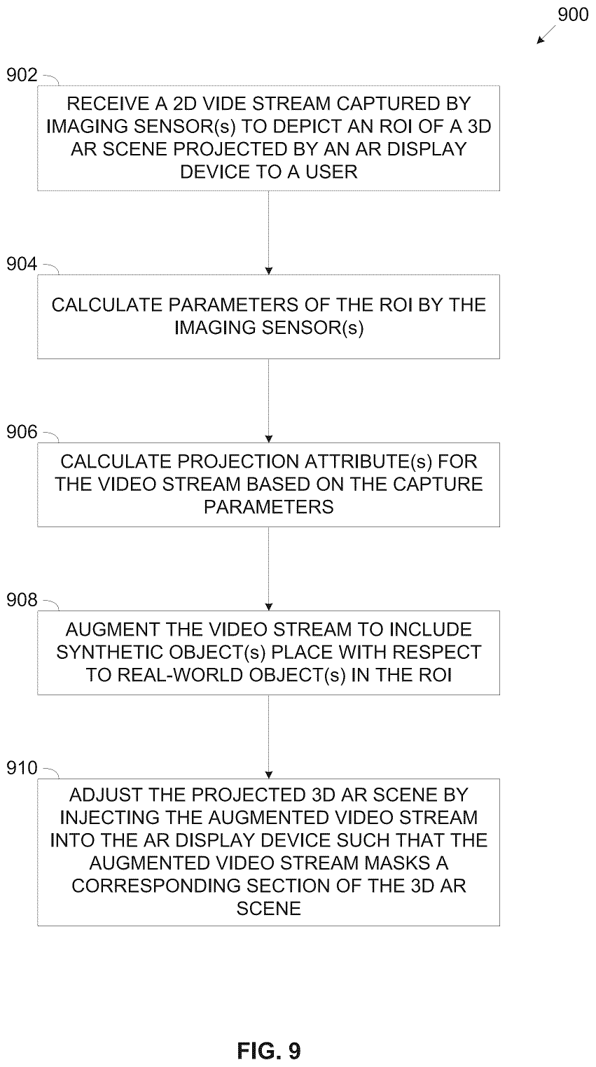

Reference is now made to FIG. 9, which is a flowchart of an exemplary process of enhancing accuracy of an Augmented Reality (AR) scene, according to some embodiments of the present invention.

An exemplary process 900 may be executed to enhance accuracy of an AR scene by increasing accuracy of positioning one or more synthetic objects with respect to the position of one or more real-world objects depicted in the AR scene.

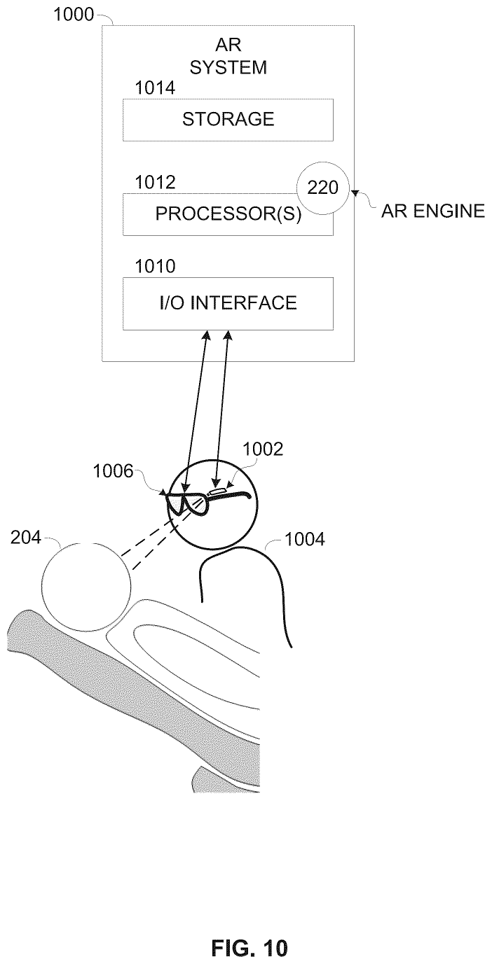

Reference is also made to FIG. 10, which is a schematic illustration of an exemplary system for enhancing accuracy of a dental AR scene, according to some embodiments of the present invention.

An AR system 1000, for example, a computer, a server, a processing node, and/or the like may be configured to execute the process 900 for enhancing an AR scene projected by one or more AR display devices 1006, for example, an HMD, AR goggles, and/or the like to one or more users 1004. Optionally, the AR system 1000 may be integrated in the AR display devices 1006.

The AR system 1000 may comprise an Input/Output (I/O) interface 1010 such as the I/O interface 210, a processor(s) 1012 such as the processor(s) 212 for executing the process 900 and a storage 1014 such as the storage 214 for storing data and/or code (program store).

Via the I/O interface 1010, the AR system 200 may connect and communicate with the AR display device(s) 1006. The AR system 200 may further communicate, via the I/O interface 1010, with one or more imaging sensors 1002 such as the imaging sensor 202.

The imaging sensor(s) 1002 may be deployed and positioned in line with a line of sight of the user 1004 such that a view angle (viewpoint) of the imaging sensor(s) 1002 is aligned with the view angle of the eyes of the user 1004. For example, the imaging sensor(s) 1002 may be attached, integrated, connected and/or otherwise coupled to the AR display device 1006 used (worn) by the user 1004 the imaging sensor(s) 1002 move with the head of the user 1004 thus aligned to the line of sight of the user 1004.

As described herein before for the processor(s) 212, the processor(s) 1012 may execute one or more functional modules implemented using one or more software modules, one or more of hardware modules available in the AR system 200 and/or combination thereof. For example, the processor(s) 1012 may execute an AR engine 920 configured to execute the process 900 for enhancing the AR scene projected by the AR display device 1006 to the user 1004.

According to some embodiments, the AR session is a dental treatment session in which the user 1004 is a dental caregiver treating a patient such as the patient 204. The dental caregiver 1004 may use the AR display device to visualize one or more dental anatomy features extracted from a dental 3D CT model of the patient 204, for example, a tooth root, a root canal and/or the like. In such embodiments, one or more of the extracted dental anatomy features may be inserted as synthetic objects into the AR scene projected to the dental caregiver 1004 and positioned accurately with respect to one or more visible dental features the dental caregiver 1004 sees in the AR scene. The dental care giver 1004 analyzing the AR scene comprising the added dental anatomy feature(s) may thus assess, treat and/or operate the patient 204 accordingly with significantly extended view and perspective of the intraoral environment of the patient 204.

As shown at 902, the process 900 starts with the AR engine 920 receiving a video stream, specifically a 2D video stream captured by the imaging sensor(s) 1002 aligned with the line of sight of the user 1004.

Since the imaging sensor(s) 1002 is positioned in line with the eyes of the user 1002, the video stream may depict a certain ROI positioned in front of the user 1004, i.e., perpendicularly to the line of sight of the user 1004.

As shown at 904, the AR engine 920 may calculate one or more (operational) parameters of the imaging sensor(s) 1002 which may impact the capture of the video stream in order to identify possible shifts, errors and/or the like in one or more frames of the video stream captured by the imaging sensor(s) 1002 compared to the scene viewed by the user 1002.

The parameters of the imaging sensor(s) 1002 may comprise one or more intrinsic parameters such as, for example, a focal length, a sensor format, a principal point, and/or the like and/or one or more extrinsic parameters such as, for example, a position of the imaging sensor 202 with respect to the eyes of the user 1004, a field of view and/or the like.

The calculation of the intrinsic and/or extrinsic parameters of the imaging sensor(s) 202 may be typically done during calibration of the imaging sensor(s) 202 as known in the art prior to the AR session. However, the AR engine 920 may apply the same computation methods and/or algorithms known in the art to re-calculate and/or correct these parameters during the AR session to adjust according to potential changes in the parameters of the imaging sensor(s) 1002.

As shown at 906, the AR engine 920 may calculate, based on the operational parameters of the imaging sensor(s) 1002, one or more projection attributes for projecting one or more of the frames of the video stream to the user 1004 via the AR display device.

Moreover, in order to increase positioning and/or alignment accuracy of the 2D video stream with respect to the 3D AR scene, the AR engine 920 may further adjust one or more of the projection attributes according to one or more display parameters of the AR display device, for example, a format, an orientation, a resolution, a positioning and/or the like.

As shown at 908, the AR engine 920 may augment one or more of the frames of the video stream by inserting into the video stream one or more synthetic objects positioned with respect to one or more real-world objects depicted in the video stream according to the calculated projection attribute(s).

As shown at 910, the AR engine 920 may adjust the AR scene projected by the AR display device 1006 to the use 1004 by injecting the augmented video stream into the display of the AR display device 1006.

In particular, the AR engine 920 may inject the augmented video stream directly into the display of the AR display device 1006 such that the augmented video stream masks and conceals a corresponding section of the 3D AR scene.

Since the synthetic object(s) are inserted into the 2D video stream, there may be significantly less degrees of freedom for positioning the synthetic object(s) compared to the number of degrees of freedom existing in inserting synthetic object(s) to a 3D AR scene. The positioning of the synthetic object(s) in the frames of the video stream with respect to real-world object(s) depicted in the frames may be therefore significantly more accurate compared to positioning such synthetic object(s) in the 3D AR scene as may be done by existing methods and system.

Moreover, since both the synthetic object(s) and their reference real-world object(s) are displayed in the AR scene as part of the augmented video stream, the positioning of the synthetic object(s) with respect to the reference real-world is maintained as opposed to injecting 3D synthetic object(s) into the 3D AR scene as may be done by the existing methods. Therefore, even if the augmented video stream is shifted with respect to the AR scene, the synthetic object(s) and their reference real-world object(s) which are part of the augmented video stream may not shift with respect to each other thus accurately positioned with respect to each other.

Optionally, the AR engine 920 may apply one or more smoothing algorithms as known in the art for smoothing one or more edges of the augmented video stream injected into the 3D AR scene. Smoothing the edges of the augmented video stream to make it better blend with the AR scene may improve the user experience and/or user impression of the 3D AR scene augmented to include the 2D augmented video stream which replaces the corresponding section of the AR scene.

Optionally, the AR engine 920 may adjust a zoom of one or more frames of the video stream before injecting it into the AR scene. The AR engine 920 may adjust the zoom of the frame(s) before and/or after augmenting them to include the added synthetic object(s).

Optionally, the AR engine 920 may adjust one or more attributes of one or more pixels of one or more frames of the video stream before injecting it into the AR scene, for example, brightness, color, contrast, gamma correction and/or the like. as such, one or more of the objects, specifically the real-world objects depicted in the frame(s) may be brightened up, darkened, sharpened, re-colored and/or the like.

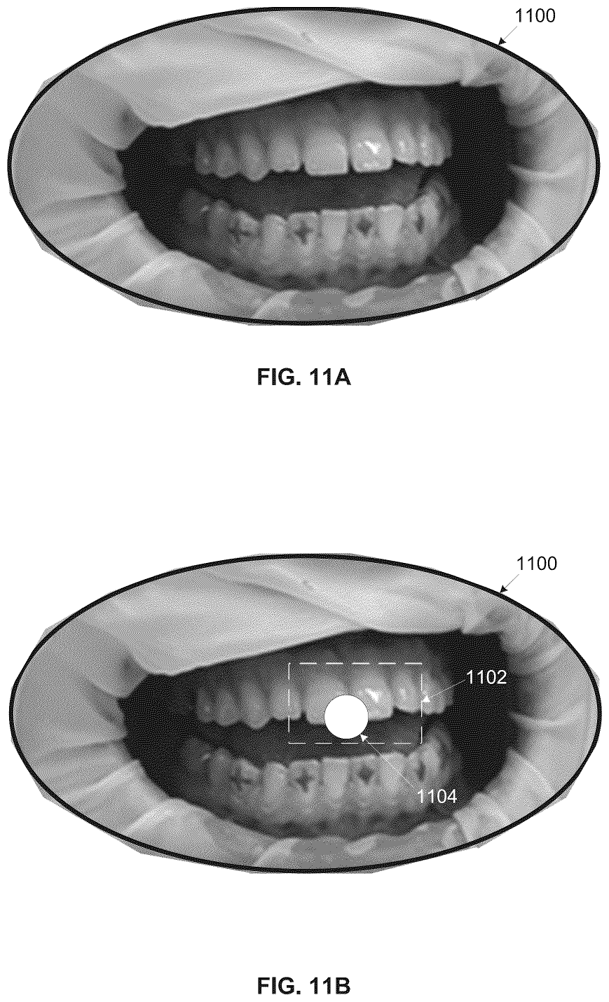

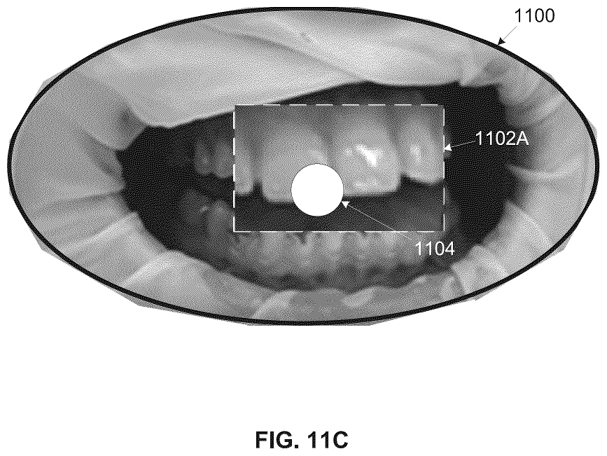

Reference is now made to FIG. 11A, FIG. 11B and FIG. 11Cm which are schematic illustrations of an exemplary dental AR scene augmented to include by injecting an augmented video stream in which synthetic object is positioned accurately with respect to real-world objects depicted in the video stream, according to some embodiments of the present invention.

As seen in FIG. 11A, an exemplary AR scene 1100 may be a dental AR scene projected by an AR display device such as the AR display device 1006 to a dental care giver such as the user 1004 providing a dental treatment to a patient such as the patient 204.

As seen in FIG. 11B, an AR engine such as the AR engine 920 may augment the dental AR scene 1100 by injecting one or more augmented frames 1102 of a video stream which an exemplary synthetic object 1104 is added and positioned accurately with respect to one or more real-world depicted in the frame 1104 as described in the process 900, for example, one or more teeth of the patient 204. Note that the dashed line marking the edges of the augmented frame 1102 is only presented to indicate the augmented frame 1102 and is not displayed in the actual AR scene.

As seen in FIG. 11C, the AR engine 920 may optionally adjust a zoom of one or more frames 1104 of the video stream, for example zoom-in to produce a zoom-in frame 1104A in which the synthetic object 1104 is positioned accurately with respect to the teeth of the patient 204.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant systems, methods and computer programs will be developed and the scope of the terms imaging sensor, intraoral scanner, CT scanner, registration algorithm, ML model, and neural network are intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, an instance or an illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The word “exemplary” is used herein to mean “serving as an example, an instance or an illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of registering intraoral frames of a patient to a dental Computed Tomography (CT) model of the patient, comprising:
using at least one processor configured for:
receiving a dental 3D visual model of a patient created based on a visible light spectrum intraoral scan of the patient captured after at least one of a plurality of teeth of the patient is marked with a plurality of intraoral markers located inside an intraoral space of the patient;
registering the dental 3D visual model to a dental 3D CT model of the patient;
receiving a plurality of frames captured during a dental treatment to the patient, each of the plurality of frames depicts at least some of the plurality of intraoral markers;
registering each frame to the dental 3D visual model based on the at least some intraoral markers;
registering each frame to the dental 3D CT model based on the registration of the respective frame to the dental 3D visual model and the registration of the dental 3D visual model to the dental 3D CT model; and
creating a plurality of fused frames each merging a respective one of the plurality of frames with a corresponding segment of the registered dental 3D CT model.

2. The method of claim 1, wherein the dental 3D CT model of the patient is created in advance before the at least one tooth is marked with the plurality of intraoral markers.

3. The method of claim 1, wherein the plurality of intraoral markers are visible in visible light spectral region detectable by at least one imaging sensor used during the dental treatment to capture the plurality of frames.

4. The method of claim 1, wherein the plurality of frames are black and white frames and/or color frames.

5. The method of claim 1, wherein the plurality of frames comprise depth data.

6. The method of claim 1, wherein the dental 3D visual model is created based on an intraoral scan captured prior to the dental treatment after the at least one tooth is marked with the plurality of intraoral markers.

7. The method of claim 1, wherein the dental 3D visual model is created based on an intraoral scan captured during the dental treatment.

8. The method of claim 1, wherein the plurality of intraoral markers are created on the at least one tooth using at least one material approved for intra oral use.

9. The method of claim 1, further comprising registering at least one of the plurality of frames to the dental 3D CT model by registering the at least one frame to another frame previously registered to the dental 3D CT model.

10. The method of claim 1, wherein the plurality of fused frames are displayed via at least one Augmented Reality (AR) device used by a dental caregiver treating the patient.

11. A system for registering intraoral frames of a patient to a dental Computed Tomography (CT) model of the patient, comprising:
at least one processor configured to execute a code, the code comprising:
code instructions to receive a dental 3D visual model of a patient created based on a visible light spectrum intraoral scan of the patient captured after at least one of a plurality of teeth of the patient is marked with a plurality of intraoral markers located inside an intraoral space of the patient;
code instructions to register the dental 3D visual model to a dental 3D CT model of the patient;
code instructions to receive a plurality of frames captured during a dental treatment to the patient, each of the plurality of frames depicts at least some of the plurality of intraoral markers;
code instructions to register each frame to the dental 3D visual model based on the at least some intraoral markers;
code instructions to register each frame to the dental 3D CT model based on the registration of the respective frame to the dental 3D visual model and the registration of the dental 3D visual model to the dental 3D CT model; and
code instructions to create a plurality of fused frames each merging a respective one of the plurality of frames with a corresponding segment of the registered dental 3D CT model.

12. A method of registering intraoral frames of a patient to a dental Computed Tomography (CT) model of the patient, comprising:
using at least one processor configured for:
receiving a dental 3D CT model of a patient created based on a CT scan captured after at least one of a plurality of teeth of the patient is marked with a plurality of intraoral markers located inside an intraoral space of the patient, the plurality of markers are applied using at least one material having a density deviating by predefined value from the density of the plurality of teeth such that the plurality of intraoral markers are distinguishable from the plurality of teeth in the CT scan and are visualized accordingly in the dental 3D CT model;
receiving a plurality of frames captured by at least one imaging sensor during a dental treatment to the patient, each of the plurality of frames depicts at least some of the plurality of intraoral markers which are visible to the at least one imaging sensor;
registering each frame to the dental 3D CT model based on the at least some intraoral markers; and
creating a plurality of fused frames each merging a respective one of the plurality of frames with a corresponding segment of the registered dental 3D CT model.

13. A system for registering intraoral frames of a patient to a dental Computed Tomography (CT) model of the patient, comprising:
at least one processor configured to execute a code, the code comprising:
code instructions to receive a dental 3D CT model of a patient created based on a CT scan captured after at least one of a plurality of teeth of the patient is marked with a plurality of intraoral markers located inside an intraoral space of the patient, the plurality of intraoral markers are applied using at least one material having a density deviating by predefined value from the density of the plurality of teeth such that the plurality of intraoral markers are distinguishable from the plurality of teeth in the CT scan and are visualized accordingly in the dental 3D CT model;
code instructions to receive a plurality of frames captured by at least one imaging sensor during a dental treatment to the patient, each of the plurality of frames depicts at least some of the plurality of intraoral markers which are visible to the at least one imaging sensor;
code instructions to register each frame to the dental 3D CT model based on the at least some intraoral markers; and
code instructions to create a plurality of fused frames each merging a respective one of the plurality of frames with a corresponding segment of the registered dental 3D CT model.

14. A method of registering intraoral frames of a patient to a dental Computed Tomography (CT) model of the patient, comprising:
using at least one processor configured for:
obtaining at least one Machine Learning (ML) model trained to register intraoral images of a patient to a dental 3D CT model of the patient, the at least one ML model is trained with a plurality of intraoral images of the patient captured prior to a dental treatment to the patient;
receiving a plurality of intraoral frames of the patient captured by at least one imaging sensor during a dental treatment to the patient;
applying the at least one trained ML model to register each of the plurality of intraoral frames to the dental 3D CT model; and
creating a plurality of fused frames each merging a respective one of the plurality of intraoral frames with a corresponding segment of the registered dental 3D CT model.

15. A system for registering intraoral frames of a patient to a dental Computed Tomography (CT) model of the patient, comprising:
at least one processor configured to execute a code, the code comprising:
code instructions to obtain at least one Machine Learning (ML) model trained to register intraoral images of a patient to a dental 3D CT model of the patient, the at least one ML model is trained with a plurality of intraoral images of the patient captured prior to a dental treatment to the patient;
code instructions to receive a plurality of intraoral frames of the patient captured by at least one imaging sensor during a dental treatment to the patient;
code instructions to apply the at least one trained ML model to register each of the plurality of intraoral frames to the dental 3D CT model; and
code instructions to create a plurality of fused frames each merging a respective one of the plurality of intraoral frames with a corresponding segment of the registered dental 3D CT model.

16. A method of enhancing accuracy of an augmented reality (AR) scene, comprising:
using at least one processor configured for:
receiving a 2D video stream depicting a region of interest (ROI) of a AR scene projected in 3D to a user by an AR display device, the video stream is captured by at least one imaging sensor deployed such that a view angle of the at least one imaging sensor is aligned with the view angle of the user's eyes;
calculating at least one projection attribute for the video stream based on at least one parameter of the at least one imaging sensor;
augmenting at least one frame of the video stream by inserting at least one synthetic object, the at least one synthetic object is positioned with respect to at least one real-world object depicted in the at least one frame according to the least one projection attribute; and
adjusting the projected AR scene by injecting the augmented video stream into the AR display device such that the augmented video stream masks a corresponding section of the 3D AR scene.

17. The method of claim 16, wherein the AR session is a dental AR session in which the AR scene is an intraoral AR scene of a patient.

18. The method of claim 16, wherein the at least one parameter comprises at least one intrinsic parameter of the at least one imaging sensor, the at least one intrinsic parameter is a member of a group comprising: a focal length, a sensor format, and a principal point.

19. The method of claim 16, wherein the at least one parameter comprises at least one extrinsic parameter of the at least one imaging sensor, the at least one extrinsic parameter is a member of a group comprising: a position of the at least one imaging sensor with respect to the user's eyes, and a field of view.

20. The method of claim 16, further comprising applying at least one algorithm to smooth an edge of the augmented video stream injected into the 3D AR scene.

21-23. (canceled)
